(19) United States
(12) Patent Application Publication
(10) Pub. No.: US 2012/0225063 A1
(43) Pub. Date: Sep. 6, 2012

(54) TREATMENT RESPONSE TO ANTI-ANGIOGENIC THERAPIES

(76) Inventor: John K. Chan, Foster City, CA (US)

(21) Appl. No.: 13/412,610

(22) Filed: Mar. 5, 2012

Related U.S. Application Data
(60) Provisional application No. 61/449,671, filed on Mar. 5, 2011.

(51) Int. Cl. A61K 39/395; C12Q 1/02; C40B 30/00; C12Q 1/68; A61P 35/00; A61P 9/00

(52) U.S. Cl. 424/133.1; 506/7; 435/6.1; 435/29

(57) ABSTRACT

Methods are provided for selecting and treating patients with an anti-angiogenic agent and methods for reducing the risk of adverse events in patients from treatment with an anti-angiogenic agent, comprising the steps: obtaining a sample of tumor and normal tissues from a patient; determining the miRNA or protein expression in said samples; comparing the miRNA or protein expression in said tumor sample to the miRNA or protein expression in normal tissue; determining whether said tumor miRNA or protein expression is higher or lower than said normal miRNA or protein expression, wherein if said miRNA or protein expression indicates that the expression of at least one angiogenic gene is upregulated, the patient is scheduled for treatment with an anti-angiogenic agent. Preferably the anti-angiogenic agent is a VEGF-targeting agent or a tyrosine kinase inhibitor. In preferred embodiments, the VEGF-targeting agent is bevacizumab.

### CROSS-REFERENCE TO RELATED APPLICATIONS

This application claims priority from U.S. Provisional Patent Application No. 61/449,671, filed Mar. 5, 2011, which is incorporated herein in its entirety.

### FIELD OF THE INVENTION

This invention relates generally to methods and compositions for treatment of diseases such as cancer, and the like.

### BACKGROUND OF THE INVENTION

Tumor growth and metastasis are believed to be angiogenesis-dependent, providing the possibility that inhibition of new vessel formation can serve as a strategy to interfere with tumor growth and progression. The dependence of tumor growth on neovascularization has been firmly established by extensive experimental evidence, demonstrating that tumors as small as a few cubic millimeters in size are not able to continue to grow without vigorously inducing new blood vessel formation. Tumor starvation through interference with tumor blood supply has become a well-recognized approach of cancer therapy and it is hoped that targeting the tumor microenvironment can improve the efficacy of anti-cancer therapies.¹

Angiogenesis is regulated via the actions of a large number of pro-angiogenic and anti-angiogenic factors. Endogenous angiogenesis inhibitors may be able to curb tumor growth when there is an equivalent rate of cell death. However, once the effect of pro-angiogenic molecules is no longer balanced by that of anti-angiogenic factors, the angiogenic “switch” is turned on and the vascular phase of tumor growth is initiated. Pro-angiogenic and anti-angiogenic regulators may be derived from cancer cells but also from stromal cells, circulating inflammatory cells, and the extracellular matrix. Numerous angiogenic growth factor receptor pathways have been identified to date; however, the vascular endothelial growth factor (VEGF) family of proteins and receptors has been the major focus of targeted drug development in oncology.

In particular, the humanized anti-VEGF antibody bevacizumab (also known as AVASTIN®) was the first anti-angiogenic compound that was approved by the US Food and Drug Administration for use in conjunction with standard chemotherapy in advanced colorectal cancer (CRC) patients.⁸ In a prospective randomized trial of ovarian cancer patients, these investigators found an improved progression-free survival of 18.1 months with bevacizumab and chemotherapy compared to only 14.5 months with standard therapy alone added, with respective median overall survival of 28.8 and 36.6 months. (Perren et al., 2011) In the high risk patients, there was an overall survival benefit of nearly 8 months associated with bevacizumab. However, bevacizumab was associated with more toxic effects⁴⁶Likewise, Burger et al. reported a similar extension of the progression-free survival time of approximately 6 months when bevacizumab was combined with chemotherapy to treat advanced ovarian cancer.⁴⁷

Anti-vascular agents have been shown to have clinical activity, but with associated toxicities and significant costs. Bevacizumab has been shown to be associated with serious adverse events, including bowel perforation, hypertension, and heart failure.

In a recent meta-analysis, bevacizumab in combination with chemotherapy or biological therapy, compared with chemotherapy alone, was associated with increased treatment-related mortality.² Therefore, even though bevacizumab provides potent anti-vascular capabilities, its toxicities and increased incidence of mortality renders its use in patients uncertain. Moreover, the high cost in conjunction with its toxicity also limits its clinical utility.

Accordingly, there is a need for individualizing cancer therapy by selecting treatment that not only improves response and survival, but also limits toxicity. The individualization of anti-cancer therapy may also prevent the exposure of patients to unnecessary toxicity from using ineffective drugs with risk of developing drug resistance, and inappropriate use of limited healthcare resources.

### SUMMARY OF THE INVENTION

Methods are disclosed for selecting a patient for treatment with an anti-angiogenic agent. The methods comprise the steps of obtaining samples of tumor and normal tissues from a patient, determining the miRNA or protein expression in the samples, comparing the miRNA or protein expression for each miRNA or protein in the sample of tumor tissue to the miRNA or protein expression in the sample of normal tissue to determine whether tumor tissue miRNA or protein expression is higher or lower than normal tissue miRNA or protein expression. If the miRNA or protein expression indicates that the tumor tissue exhibits upregulated angiogenesis, the patient is scheduled for treatment with said anti-angiogenic agent. The methods can further comprise treating the patient with an anti-angiogenic agent. In some embodiments, the expression of miR-378, miR-214 or miR-21 is low in said sample of tumor tissue relative to normal tissue. In some embodiments, the expression of miR-128 or miR-194 is high in the sample of tumor tissue relative to normal tissue. In other embodiments, the expression of VEGF-C is high in the sample of tumor tissue relative to normal tissue. The methods are not limited to any particular cancers, and include brain, breast, kidney, pancreatic, lung, prostate, colorectal, and ovarian cancers. The anti-angiogenic agents include VEGF-targeting agents (such as bevacizumab), tyrosine kinase inhibitors or other biologic therapy.

Methods are disclosed for treating a patient suffering from cancer with an anti-angiogenic agent. The methods comprise obtaining samples of tumor and normal tissues from a patient, determining the miRNA or protein expression for angiogenic genes in the samples, comparing the miRNA or protein expression for angiogenic genes for each miRNA or protein in the sample of tumor tissue to the miRNA or protein expression in the sample of normal tissue. If the miRNA or protein expression indicates that the expression of at least one angiogenic gene is upregulated in tumor tissue, the patient is treated with an anti-angiogenic agent. The anti-angiogenic agent can be selected from a VEGF-targeting agent, a tyrosine kinase inhibitor or other biologic therapy. In some embodiments, the expression of miR-378, miR-214 or miR-21 is low in said sample of tumor tissue relative to normal tissue. In some embodiments, the expression of miR-128 or miR-194 is high in the sample of tumor tissue relative to normal tissue. In yet other embodiments, the expression of VEGF-C is high in the sample of tumor tissue relative to normal tissue. The angiogenic gene can be selected from bone morphogenetic protein 2 (BMP2), mitogen-activated protein kinase 1 (MAPK1), or Cas-Br-M ecotropic retroviral transforming sequence (CBL).

Yet other embodiments provide methods for reducing the risk of adverse events from treatment with an anti-angiogenic agent in a population of patients suffering from cancer comprising obtaining a sample of normal tissue and a sample of tumor tissue from a patient; determining the miRNA expression for angiogenic genes in said samples; comparing said miRNA expression for said angiogenic genes in said tumor sample to levels of miRNA expression for said angiogenic genes in normal tissue to determine whether said miRNA expression indicates that the expression of angiogenic genes in tumor tissue is upregulated relative to normal tissue; wherein if said miRNA expression indicates that the expression of at least one angiogenic gene is upregulated, the patient is scheduled for treatment with an anti-angiogenic agent. When the tumor miRNA expression indicates that the angiogenic genes are not upregulated relative to normal tissue, the patient is not treated with said anti-angiogenic agent, thereby avoiding unnecessary adverse events in the patient, and instead, alternative therapy is chosen. Preferably, the anti-angiogenic agent is a VEGF-targeting agent, preferably bevacizumab, or a tyrosine kinase inhibitor. The cancer includes brain, breast, pancreatic, lung, kidney, prostate, colorectal, and ovarian cancer, but is not limited to these. When the tumor miRNA expression indicates that miR-378, miR-214 or miR-21 is low, or if miR-128 or miR-194 is high in said tumor sample relative to said normal sample, treatment comprises an anti-angiogenic agent.

In yet other embodiments, methods are provided for reducing the risk of adverse events from treatment with an anti-angiogenic agent in a patient suffering from ovarian cancer comprising obtaining a sample of normal tissue and a sample of ovarian cancer tissue from the patient; determining the miRNA expression for angiogenic genes in said samples; comparing said miRNA expression for said angiogenic genes in said ovarian cancer sample to levels of miRNA expression for said angiogenic genes in normal tissue to determine whether said miRNA expression indicates that the expression of angiogenic genes in ovarian cancer tissue is upregulated relative to normal tissue; wherein if said miRNA expression indicates that the expression of at least one angiogenic gene is upregulated, the patient is scheduled for treatment with an anti-angiogenic agent. Preferably, miR-378 is used to determine whether the angiogenic genes are upregulated. Preferably, the angiogenic gene is selected from bone morphogenetic protein 2 (BMP2), mitogen-activated protein kinase 1 (MAPK1), or Cas-Br-M ecotropic retroviral transforming sequence (CBL). When the tumor miRNA expression indicates that the angiogenic genes are not upregulated relative to normal tissue, the patient is not treated with said anti-angiogenic agent, thereby avoiding unnecessary adverse events in the patient, and instead, alternative therapy is chosen. When the tumor miRNA expression indicates that miR-378 is low in said tumor sample relative to said normal sample, treatment comprises an anti-angiogenic agent. Preferably, the anti-angiogenic agent is a VEGF-targeting agent, preferably bevacizumab, or a tyrosine kinase inhibitor. The cancer includes brain, breast, pancreatic, lung, kidney, prostate, colorectal, and ovarian cancer, but is not limited to these.

Methods are further disclosed for reducing the risk of adverse events from treatment with an anti-angiogenic agent in a population of patients suffering from cancer. The methods comprise obtaining a sample of normal tissue and a sample of tumor tissue from a patient, determining the expression of miRNA or protein in said samples, comparing the expression of each miRNA or protein in the tumor sample to levels of expression of each miRNA or protein in normal tissue to determine whether the miRNA or protein expression is elevated in tumor tissue relative to normal tissue. If one of the following expression of miRNA or protein is found, the patient is scheduled for treatment with an anti-angiogenic agent: miR-378, miR-214 or miR-21 is low in said tumor sample relative to said normal sample; miR-128 or miR-194 is high in the tumor sample relative to the normal sample; VEGF-C expression is high in the tumor sample relative to the normal sample; complement inhibitor (such as CD55) expression is high in the tumor sample relative to the normal sample; or inflammatory chemokine (such as CCL2) expression is low in the tumor sample relative to the normal sample.

In addition, demographic and clinical factors such as treatment free interval in conjunction with molecular markers (miRNA) can be employed to further refine the selection of patients for treatment with anti-vascular agents.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 illustrates the progression-free survival after bevacizumab and chemotherapy (BC) for two treatment-free intervals greater or less than 1 month prior to initiation of BC.

FIG. 2 illustrates the progression-free survival after bevacizumab and chemotherapy based on miR-378 expression.

FIG. 3 illustrates the progression-free survival of patients after treatment with bevacizumab based on the expression of VEGF-A, -B, or -C.

FIG. 4 illustrates the survival of patients based on the expression of VEGF-A, -B, or -C, respectively.

### DETAILED DESCRIPTION OF THE INVENTION

### I. Definitions and Overview

Before the present invention is described in detail, it is to be understood that unless otherwise indicated this invention is not limited to specific drugs or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “an anti-angiogenic agent” includes two or more anti-angiogenic agents, and so forth.

“MicroRNAs” (often abbreviated as “miRNAs”) are small, non-coding single-stranded RNAs that regulate gene expression by directing the degradation of mRNA (messenger RNA), thereby inhibiting the protein translation of mRNA transcripts. More specifically, miRNAs are transcribed into long primary transcripts called pri-miRNAs that are subsequently processed into smaller hairpin precursors of 60-70 nucleotides called pre-miRNA, by an RNAse III enzyme, Drosha. Specific miRNAs are referred to by number, for example miRNA-378 or miR-378.

As used herein, the terms “anti-angiogenic” and “anti-vascular” may be used interchangeably herein.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The present inventor recognized a need to prevent exposure of patients to ineffective drugs with resulting toxicities and adverse events, as well as the need to avoid development of drug resistance when the tumor cells are not treated effectively using a particular agent. Individualizing therapy for specific patients is desired so that appropriate drugs can be used where they will provide the maximum benefit to patients, and not further increase morbidity and mortality for these fragile patients. In addition, due to the high cost of some of these agents, there is concern that their use is unjustified in view of limited healthcare resources.

The present inventor recognized that the ability to predict for response to anti-vascular agents and personalize cancer therapy could be found in the patient's own regulatory miRNA or protein expression (determined by mRNA) profiles. In particular, it was discovered that miRNA can be utilized as a novel predictor for response to anti-vascular agents (e.g., anti-VEGF antibodies) in cancer patients (e.g., recurrent serous ovarian cancer). More specifically, using miRNA profiles to determine patient tumor angiogenesis status provides important new information enabling individualized therapy with anti-vascular agents such as bevacizumab. In addition, the present inventor has surprisingly discovered that the regulation of the patient's tumor's own angiogenic apparatus is critical in determining whether the patient's prognosis to treatment with bevacizumab is favorable or not efficacious.

The miRNA profiles or protein expression profiles of tumor tissues and normal tissues derived from patients were analyzed. In Example 1, it is demonstrated that low expression levels of miR-378 confer improved survival after treatment with bevacizumab plus chemotherapy, presumably due to more effective targeting of upregulated angiogenesis in this class of patients. Low miR-214, high miR-128, and low miR-21* were also associated with a higher 6 month progression-free survival after bevacizumab plus chemotherapy, while low miR-194, was correlated with lower progression-free survival after bevacizumab plus chemotherapy.

In Example 2, it is demonstrated that increased tumor tissue expression of VEGF-C (in comparison with the patient's own normal tissue) is associated with poor prognosis in treatment with anti-angiogenesis agents. Patients with high expression of VEGF-C had worse overall survival than those with low VEGF-C. In a subset analysis of those who underwent treatment with an anti-vascular agent, there was a suggestion that higher expression of VEGF-C was associated with poorer response to anti-vascular agents.

Genes associated with immune regulation can have effects on tumor invasiveness and angiogenesis. For example, inflammatory cytokines such as CCL2 induce endothelial cell migration, sprouting and tube formation, which aid tumor angiogenesis. Complement inhibitors such as CD55 reduce cell mediated cytotoxicity of tumors. In Example 3, immune regulatory genes were screened for level of expression (tumor tissue relative to normal tissue controls) and correlated with efficacy of treatment with bevacizumab. Out of 69 genes screened, a low expression of the inflammatory chemokine CCL2 and high expression of the complement inhibitor CD55 were associated with longer progression-free survival after bevacizumab and chemotherapy. Kaplan-Meier estimates showed patients with low CCL2 had a 6-month and median progression-free survival of 68% and 8.7 months compared to 20% and 2.9 months for patients with high CCL2. Patients with high CD55 had a 6-month progression-free survival of 59% and 8.7 months median vs. 25% and 3.0 months for patients with low CD55. Tumors expressing CD55 had a decreased risk of progression when the patient was treated with bevacizumab and chemotherapy.

### II. Anti-Vascular Agents

Anti-angiogenic therapies using anti-VEGF neutralizing antibodies have been shown to target the VEGF system in cancer patients.^{3,4} Although most available information is on the monoclonal antibody bevacizumab, there are other agents such as the small molecule tyrosine kinase inhibitors that have tropism for the ATP-binding site of the VEGF receptor KDR/Flk-1, and therefore act as inhibitors of VEGF receptor (VEGFR) signaling.⁵ Other methods for inhibiting VEGF include the use of soluble receptors and ribozymes, and combined approaches to inhibiting receptor tyrosine kinases.

Non-VEGF-based angiogenesis methods such as inhibition of hypoxia-inducible factors, cox-2 inhibitors, and tyrosine kinase inhibitors, as well as vascular disrupting agents, are also of interest.⁶ Anti-angiogenic agents that target other angiogenesis signaling pathways such as platelet-derived growth factor-C (PDGF-C), Bombina variagata peptide 8 (By8, also known as prokineticin-2), and VEGFR-3 are also exemplary anti-angiogenic agents.⁷ Fusion proteins (such as AMG 386) have also been constructed as anti-angiogenic agents. AMG 386 is a “peptibody” composed of a peptide that binds angiopoietins Ang-1 and Ang-2, fused with a human IgG1 Fc, and selectively inhibits the interactions of Ang-1 and Ang-2 with their receptor, Tie2, thereby affecting angiogenesis.⁴⁴ In combination with chemotherapy, AMG 386 exhibited evidence of anti-tumor activity and a dose-response effect.⁴⁵

### A. VEGF-Targeting Agents

VEGF-targeting agents include, but are not limited to, anti-VEGF antibodies. Any antibody that targets VEGF and thereby reduces its biological activity in vivo can be utilized. The anti-VEGF antibody, bevacizumab, targets VEGF-A. Antibodies that interfere with VEGF binding to its receptor can also be considered VEGF targeting agents. Ramucirumab and IMC-18F1 are exemplary monoclonal antibodies that target the VEGF receptors VEGFR-2 and VEGFR-1, respectively. Aflibercept (VEGF-Trap), a peptide-antibody fusion targeting VEGF ligand, is being tested in clinical trials.⁷

### B. Anti-Angiogenic Tyrosine Kinase Inhibitors

Anti-angiogenic tyrosine kinase inhibitors (TKIs) primarily target VEGFR2 tyrosine kinase activity as the major mediator of angiogenic signaling. The newer TKI compounds under investigation and development target a broader set of receptor and non-receptor tyrosine kinases due to the enhanced understanding of the complexity of angiogenesis regulation, and inhibit multiple VEGFRs, mostly in addition to the tyrosine kinases of other important signaling pathways (e.g. PDGFR and EGFR). Useful TKI compounds include, but are not limited to, PTK/ZK (Vatalanib); BAY 43-9006, Sorafenib (NEXAVAR®); SU 11248, Sunitinib (SUTENT®); ZD 6474 (ZACTIMA™); AEE 788; Imatinib (GLIVEC®); Gefitinib (IRESSA™); Erlotinib (TARCEVA™); brivanib; pazapanib; axiotinib; cedaranib; and cabozantinib; and the like. These small-molecule TKIs may also cause significant toxicity. Hypertension is a commonly observed grade ¾ adverse event among anti-angiogenic agents. However, the adverse effects frequently associated with administration of anti-VEGF antibody bevacizumab (e.g. hemorrhage or arterial thromboembolic events) were not found to be generally increased in first clinical trials involving anti-angiogenic TKIs.⁸

### III. Cancers

The present methods are potentially useful in a wide range of malignancies. In particular, solid tumors are dependent on vascularization for nutrient supply and optimal growth. Angiogenesis results in the growth of new blood vessels and enhanced vascular supply, and can act to effectively sustain or expand the tumor cell population. Any cancer whose growth and metastasis is mediated by enhanced vascularization is included in the instant methods.

In particular embodiments, the methods are useful in the treatment of patients suffering from brain, breast, pancreatic, lung, prostate, colorectal, kidney, and ovarian cancers, although this list is merely exemplary and not intended to be limiting.

Ovarian cancer is the most lethal gynecologic malignancy with an estimated 14,600 associated deaths and 21,500 new cases per year in the United States.⁹ Accordingly, in a preferred embodiment, the methods are useful in the treatment of patients suffering from ovarian cancer.

### IV. MicroRNAs

MicroRNAs (miRNA) are small, non-coding single-stranded RNA that regulate gene expression by directing the degradation of mRNA, thereby inhibiting the protein translation of mRNA transcripts. More specifically, miRNAs are transcribed into long primary transcripts called pri-miRNAs that are subsequently processed into smaller hairpin precursors of 60-70 nucleotides called pre-miRNA, by an RNAse III enzyme, Drosha. The pre-miRNA is exported to the cytoplasm where it is modified by another RNAse enzyme, Dicer, to result in a 19-24 nucleotide duplex. One strand is degraded, leaving the mature, single-stranded miRNA, which is then integrated into the RNA-induced silencing complex (RISC) that, depending on its degree of complementarity, either cleaves target mRNA or represses protein translation. By silencing various target mRNAs, miRNAs play key roles in diverse regulatory pathways, including control of development¹⁰, cell differentiation, apoptosis^{11,12}, cell proliferation¹³, division¹⁴, protein secretion¹⁵, viral infection^{16,17}, and cancer development.^{19,43} The regulation of miRNAs has been implicated in the survival and prediction of chemo-resistance.¹⁸

Oncogenic miRNAs promote tumor development by inhibiting known tumor-suppressor genes. MiRNAs can effectively act as oncogenes or tumor-suppressor genes, depending on the cellular context and target genes that they regulate, and can be involved in tumor progression and metastasis. MiRNAs may be implicated in tumor aggressiveness, e.g., migration, invasion, cell proliferation, epithelial-to-mesenchymal transition, angiogenesis and apoptosis. MiRNAs can be involved in multiple cellular pathways and can elicit more than one biological effect in a given cell. Thus, there is potential clinical utility of miRNAs as prognostic and predictive markers or as therapeutics for aggressive and metastatic cancers.¹⁹ MiRNA are differentially expressed in cancers and may serve as key biomarkers in the detection and prognosis of breast, pancreatic, lung, prostate, colorectal, and ovarian cancers. There have been few reports on the significance of miRNA in ovarian cancers and their role as a predictor for response to chemotherapy.^{18, 20-26}

Evidence is emerging that miRNAs are important players in endothelial cell biology and tumor angiogenesis. Dynamic changes in miRNA expression response to growth factor stimulation or hypoxia imply that miRNAs are an integral component of the angiogenic program.^{27 28}

Numerous miRNAs have been implicated in cancer. There have been several reports on the significance of miRNA in ovarian cancers and their possible role as biomarkers of treatment response. Eitan et al. showed that miR-27a, miR-23a, miR-30c, let-7g, and miR-199a-3p were upregulated in chemotherapy-resistant ovarian cancer tumors, while miR-378, and miR-625 were upregulated in tumors sensitive to chemotherapy.²⁵ In addition, Nam et al. found that high expression of miR-141, miR-18a, miR-93, and miR-429 and decreased expression of let-7b, and miR-199a were correlated with improved survival.²⁶

The present discovery relates in particular to the utility of miRNA-378, miRNA-194, miRNA-21, miRNA-214 miRNA-128, miRNA-145, and miRNA-375 in predicting treatment response to BEV.

### MiRNA-378

MiR-378 in particular was discovered by the present inventor to be a strong independent predictor for response to bevacizumab and chemotherapy (BC). To further elucidate the potential association between miR-378 and response to bevacizumab, a subset analyses was performed to determine if miR-378 was simply a prognosticator for survival and/or response to cytotoxic chemotherapy alone. To determine if miR-378 may predict overall survival, an analysis on this group was performed and it was found that miR-378 was not important in the overall survival in this subset of patients. In addition, 103 patients who received chemotherapy alone for recurrent disease were studied and it was found that miR-378 did not predict for response to cytotoxic therapy. Thus, it is likely that MiR-378 in particular is a strong independent predictor for response to bevacizumab. MiRNA-378 has been reported to promote cell survival by targeting Sufu and fus-1 and to regulate tumor angiogenesis by indirect upregulation of angiogenic factors.

The biological rationale underlying the potential association between miR-378 and response to anti-vascular therapy was explored. Using Targetscan to investigate the potential gene targets involved, it was found that miR-378 regulates BMP2 or Bone morphogenetic protein 2 and associated BMP2/Smad signaling, which is correlated with VEGF.^{29,30} In addition, miR-378 is implicated with MAPK1 or mitogen-activated protein kinase 1 with activation of angiopoietins and factor XII induced angiogenesis.^{31,32} Others have also shown that miR-378 may indirectly play a role in PI3K/Akt pathway for endothelial cell survival and fibroblast growth factor induced angiogenesis via CBL, Cas-Br-M (murine) ecotropic retroviral transforming sequence.^{33,34} It has also been shown that miR-378 regulates endothelial cell function contributing to angiogenesis.³⁵ Lee et al. demonstrated that miR-378 promotes cell survival, tumor growth, and angiogenesis by targeting tumor suppressors SuFu and Fus-1.¹⁹

### MiRNA-194

It has been proposed that miR-194, which is specifically expressed in liver parenchymal cells, may play a role in preventing liver cancer cell metastasis. MiR-194 is specifically expressed in the human gastrointestinal tract and kidney, and highly expressed in hepatic epithelial cells, but not in Kupffer cells or hepatic stellate cells, two types of mesenchymal cells in the liver. MiR-194 expression was decreased in hepatocytes cultured in vitro, which had undergone a dedifferentiation process, and in liver mesenchymal-like cancer cell lines. The overexpression of miR-194 in liver mesenchymal-like cancer cells reduced the expression of the mesenchymal cell marker N-cadherin and suppressed invasion and migration of the mesenchymal-like cancer cells both in vitro and in vivo. MiR-194 was shown to target the 3′-UTRs of several genes that were involved in epithelial-mesenchymal transition and cancer metastasis.³⁶

### MiRNA-21

MiRNA-21 is highly expressed in endothelial cells.³⁵ MiR-21 expression was higher in de-differentiated vascular smooth muscle cells in culture, and depletion of miR-21 resulted in apoptosis and decreased proliferation. MiR-21 mediated effects were confirmed in vivo in a balloon-injured rat carotid artery model.³⁷

### MiRNA-214

MiR-214 is closely related to endothelial nitric oxide synthase (eNOS) and hence is involved in angiogenesis. Ginsenoside-Rg1, one of the active components of ginseng, has been confirmed as an angiogenesis inducer. Using miRNA microarray analysis, a miR-214 was found to be down-regulated by Rg1 in human umbilical vein endothelial cells, leading to an increase in eNOS expression, and in vitro cell migration and tube formation which can possibly promote angiogenesis.³⁸

### V. Determination of miRNA Expression

Tumor tissues were derived from high quality specimens: 70% tumor nuclei, <20% necrosis, 1 cm³ tissue, and 20 μg DNA/RNA. Normal tissues neighboring the cancers were also removed and served as controls. MiRNA expression was determined using conventional laboratory techniques. In preferred embodiments, high expression of miRNAs is defined as miRNA derived from tumors having at least 1.5 times the level in patients' normal tissues, and low expression of miRNAs are defined as miRNA derived from tumors having no more than 2/3 the level in patients' normal tissues. In alternate embodiments, high expression of miRNAs are defined as miRNA derived from tumors having at least twice the level in patients' normal tissues, and low expression of miRNAs are defined as miRNA derived from tumors having half or less than half the level in patients' normal tissues.

### VI. Method for Selecting a Patient for Treatment with an Anti-Angiogenic Agent

Treatment with bevacizumab is predicated on the theory that bevacizumab interferes with VEGF mediated processes such as angiogenesis that can be growth promoting and/or enhance metastasis in cancer. While not wishing to be bound by theory, the present inventor has discovered that patients having upregulated tumor angiogenesis (as assessed using tumor miRNA expression) are most likely to benefit from anti-angiogenic therapies such as bevacizumab. This finding is in contrast to a report showing a positive association between tumor cell VEGF-A expression and progression of disease when treated with bevacizumab. These authors suggested that high levels of tumor cell VEGF-A are associated with resistance to bevacizumab, emphasizing the complexity of the tumor microenvironment.⁴²

In fact, after bevacizumab and chemotherapy treatment, 60% of patients with low miR-378 (i.e. upregulated tumor angiogenesis expression) had 6-month or greater progression-free survival whereas all patients with normal miR-378 recurred or died within 6 months.

Accordingly, the instant methods provide for selecting a patient for treatment with an anti-angiogenic agent comprising the steps: obtaining a sample of tumor and normal tissues from a patient; determining the miRNA expression in said samples; comparing the miRNA expression in said tumor sample to the miRNA expression in normal tissue to determine whether said tumor miRNA expression is higher or lower than said normal miRNA expression, wherein if said miRNA expression indicates that miR-378 is low relative to normal tissue, the patient is scheduled for treatment comprising said anti-angiogenic agent. Preferably, the patient is suffering from cancer, such as but not limited to breast, pancreatic, lung, prostate, colorectal, or ovarian cancer. Preferably the anti-angiogenic agent is a VEGF-targeting agent or a tyrosine kinase inhibitor. In preferred embodiments, the VEGF-targeting agent is bevacizumab.

In further embodiments, methods are provided for reducing the risk of adverse events from treatment with an anti-angiogenic agent in a patient suffering from cancer, including but not limited to ovarian cancer, comprising obtaining a sample of normal tissue and a sample of cancer tissue from the patient; determining the miRNA expression for angiogenic genes in said samples; comparing said miRNA expression for said angiogenic genes in said cancer sample to levels of miRNA expression for said angiogenic genes in normal tissue to determine whether said miRNA expression indicates that the expression of angiogenic genes in cancer tissue is upregulated relative to normal tissue; wherein if said miRNA expression indicates that the expression of at least one angiogenic gene is upregulated, the patient is scheduled for treatment with an anti-angiogenic agent. The cancer includes breast, pancreatic, lung, prostate, colorectal, and ovarian cancer, but is not limited to these. Preferably, the angiogenic gene is selected from bone morphogenetic protein 2 (BMP2), mitogen-activated protein kinase 1 (MAPK1), or Cas-Br-M ecotropic retroviral transforming sequence (CBL). When the tumor miRNA expression indicates that the angiogenic genes are not upregulated relative to normal tissue, the patient is not treated with said anti-angiogenic agent, thereby avoiding unnecessary adverse events in the patient, and instead, alternative therapy is chosen. Preferably, miR-378 is used to determine whether the angiogenic genes are upregulated. When the tumor miRNA expression indicates that miR-378 is low in said tumor sample relative to said normal sample, treatment comprises an anti-angiogenic agent. Preferably, the anti-angiogenic agent is a VEGF-targeting agent, preferably bevacizumab, or a tyrosine kinase inhibitor.

Surprisingly, some patients with high or low expression of miRNAs not as yet known to be related to angiogenesis also showed benefit from treatment with bevacizumab. In certain embodiments, the instant methods provide for selecting a patient for treatment with an anti-angiogenic agent comprising the steps: obtaining a sample of tumor and normal tissues from a patient; determining the miRNA expression in said samples; comparing the miRNA expression in said tumor sample to the miRNA expression in normal tissue to determine whether said tumor miRNA expression is higher or lower than said normal miRNA expression, wherein if said miRNA expression indicates that miR-378, miR-214 or miR-21 is low relative to normal tissue, or if miR-128 or miR-194 is high relative to normal tissue, the patient is scheduled for treatment comprising said anti-angiogenic agent. Preferably, the patient is suffering from cancer, such as but not limited to breast, pancreatic, lung, prostate, colorectal, or ovarian cancer. Preferably the anti-angiogenic agent is a VEGF-targeting agent or a tyrosine kinase inhibitor. In preferred embodiments, the VEGF-targeting agent is bevacizumab.

In certain other embodiments, methods are provided for treating a patient suffering from cancer with an anti-angiogenic agent, comprising obtaining a sample of normal tissue and a sample of tumor tissue from the patient; determining the miRNA expression for angiogenic genes in said samples; comparing said miRNA expression for said angiogenic genes in said tumor sample to levels of miRNA expression for said angiogenic genes in normal tissue to determine whether said miRNA expression indicates that the expression of angiogenic genes in tumor tissue is upregulated relative to normal tissue; wherein if said miRNA expression indicates that the expression of at least one angiogenic gene is upregulated in tumor tissue, the patient is treated with an anti-angiogenic agent. When the miRNA expression indicates that the angiogenic genes are not upregulated relative to normal tissue, the patient is not treated with said anti-angiogenic agent, thereby avoiding unnecessary adverse events in the patient. Preferably, the anti-angiogenic agent is a VEGF-targeting agent such as bevacizumab, or a tyrosine kinase inhibitor. The cancer includes but is not limited to breast, pancreatic, lung, prostate, colorectal, and ovarian cancer. In preferred embodiments, the miRNA expression indicates that miR-378, miR-214 or miR-21 is low, or if miR-128 or miR-194 is high in said tumor sample relative to said normal sample, indicating that treatment comprising an anti-angiogenic agent is warranted.

### VII. The Cancer Genomic Atlas Project

The Cancer Genomic Atlas (TCGA) project is an integrated network of clinical sites, core facilities, and genome characterization and sequencing resources. Initiated in 2006 by the National Cancer Institute (NCI) and the National Human Genome Research Institute (NHGRI), this comprehensive database of the molecular basis of cancer has been a highly valuable resource for both clinical and molecular research. The TCGA Project is comprised of two Biospecimen Core Resource (BCR) institutions, seven Genome Characterization Centers, three Genome Sequencing Centers, six Genome Data Analysis Centers, and a Data Coordinating Center. TCGA tumor specimens were acquired from participating institutions and processed and validated by the BCR. Numerous papers have been published, implicating the significance of miRNA in glioblastoma and breast cancer using TCGA data. Recently, TCGA made available their genomic and clinical data of over 430 serous ovarian cancer patients. These data are accessible to the public and were utilized in the Example described below.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, pharmaceutical chemistry, clinical practice and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

In the following examples, efforts have been made to ensure accuracy with respect to numbers used (e.g., amounts, temperature, etc.) but some experimental error and deviation should be accounted for.

Abbreviations:

BC bevacizumab plus chemotherapy

BEV bevacizumab

miRNA microRNA

OS overall survival

PFS progression free survival

TFI treatment free interval

TGCA The Cancer Genomic Atlas

VEGF vascular endothelial growth factor

### Example 1

### Time to Recurrence in Ovarian Cancer Patients Treated with Bevacizumab and Chemotherapy is Associated with Patient Specific miRNA Profiles

The Cancer Genome Atlas (TCGA) contains genomic data from high quality biospecimens of primary, newly diagnosed, untreated, serous ovarian or peritoneal cancer from 15 academic and/or cancer centers internationally. Most surgeries were performed at centers with gynecologic oncologists with detailed information on the extent of cytoreductive surgery. All tumors are matched with non-tumor tissue controls from the same patient. Moreover, all specimens underwent central pathology review to confirm histology cell type and specimen quality: 70% tumor nuclei, <20% necrosis, 1 cm³ tissue, and 20 μg DNA/RNA. Furthermore, detailed clinical data including adjuvant treatment with specific drugs and outcomes were available.

TCGA database was searched for ovarian cancer patients. Of 435 ovarian cancer patients, 147 recurred, with median time to recurrence of 8.6 months (range: 0-106). Of those that recurred, 103 were not treated with chemotherapy, 10 had adjuvant bevacizumab at primary treatment, and only 3 had bevacizumab alone. The remaining 34 patients who underwent bevacizumab treatment combined with chemotherapy for their recurrence were selected for study.

Tissues used were restricted to those with at least 80% tumor nuclei and at most 50% tissue necrosis. Following RNA extraction, microRNA of each specimen was profiled on Agilent microRNA array platform H-microRNA_{—}8×15 Kv2:01.

Patient clinical and tumor specimen data were abstracted from TCGA data portal at <http://tcga.cancer.gov/dataportal/data/about/> on Jul. 27, 2010. MiRNA expression data was obtained from the unc.edu_H-microRNA_{—}8×15Kv2_mirna_expression_analysis.txt file, and clinical data from the clinical_patient_all_OV.txt file from the clinical platform. Progression free survival data was not immediately available for use, however we calculated the time from the last day of one regimen to the first day of the subsequent regimen as each patient's progression free survival, with respect to the first drug therapy. This drug treatment information was obtained through the clinical_drug_all_OV.txt file in the clinical platform.

MiRNAs of interest were determined by using the Data Browser function in TCGA. Patients with PFS≧9 months after BC treatment (n=3) were considered as BC responders whereas those who had PFS<6 were consider as BC resistant. These two defined groups were used to screen for differential miRNA expression as previously described for platinum resistance.²⁵ Stringent selection criteria were used, including 2-fold differential expression of microRNA (compared to patients' normal tissue control).

Chi-squared and Kaplan-Meier survival analysis were employed to determine association with clinical factors and microRNA expression with patient prognosis.³⁹ Cox proportional hazards model was used to identify independent prognostic factors.⁴⁰ Fisher's exact test was used to correlate normal and low/high expression levels with 6-months of PFS; Bonferroni correction was used to adjust for multiple comparisons. The p-values from Fisher's Exact Test were divided by 800 adjusting for all possible microRNAs. P-values less than 0.05 after the correction denoted statistical significance. SPSS Statistics GradPack 17.0 software was used for our statistical analysis.

Targetscan 5.1 (http://targetscan.org/) was used to predict microRNA targets based on four features of the microRNA and gene target: site-type, 3′ pairing, local AU, and position, giving a context score as calculated by Grimson et al., 2007.⁴¹ Genes with the lowest context scores were used to predict the most likely targets of the microRNA.

### Results

Our study cohort contained 34 patients who received bevacizumab combined with chemotherapy (BC) for their recurrence. From a total of 435 ovarian cancer patients, 147 recurred with median time to recurrence of 8.6 months (range: 0-106). After excluding those who had chemotherapy alone (n=103), prior bevacizumab at primary treatment (n=10), and bevacizumab alone for their recurrence (n=3), the remaining 34 patients underwent bevacizumab combined with chemotherapy for their recurrence comprised of our study group. Of these 34 patients, the median age was 56 years (range: 36-76); 91% were White and 9% were of other race. The majority (91.2%) of patients were diagnosed with advanced-stage (stage III-IV) disease. Grade 3 tumors comprised 61.8% of tumors (Table 1). The median time to first recurrence was 11.3 months (range: 0.2-101.5). The 5-year overall survival (OS) was 51%±10% with a median follow-up time of 46 months. Those who had a longer treatment-free interval (TFI) (median=11.3 months) had a 5-year OS of 74%±11% vs. 21%±13% in those with shorter TFI (p=0.001). Moreover, longer TFI of last treatment prior to bevacizumab and chemotherapy (median=1 month) was associated with 1-year overall survival 80%±13% vs. 35%±16% in those with shorter TFI (p=0.027). Other clinical factors such as age, race, stage, grade, and optimal cytoreductive surgery were not associated with overall survival in this subset of patients.

TABLE 1
Demographic and clinico-pathologic characteristics (n = 34)

| | N (%) |
|---|---|
| Age | |
| <56 | 16 (47.1%) |
| ≧56 | 18 (52.9%) |
| Race | |
| White | 31 (91.2%) |
| Non-White | 3 (8.8%) |
| Stage | |
| I-II | 3 (8.8%) |
| III-IV | 31 (81.2%) |
| Grade | |
| 2 | 13 (38.2%) |
| 3 | 21 (61.8%) |
| Cytoreductive Surgery* | |
| Optimal (≦1 cm) | 17 (70.8%) |
| Suboptimal (>1 cm) | 7 (29.2%) |
| Treatment-free interval after initial adjuvant therapy | |
| (median = 11.3 months) | |
| <11 months | 16 (50%) |
| >11 months | 16 (50%) |
| Number of regimens prior to bevacizumab plus | |
| chemotherapy (median = 3) | |
| ≦3 | 17 (50%) |
| >3 | 17 (50%) |
| TFI of last treatment prior to bevacizumab plus | |
| chemotherapy (median = 1) | |
| <1 month | 11 (34.4%) |
| ≧1 month | 21 (65.6%) |

*Number does not add to 34 because of missing information

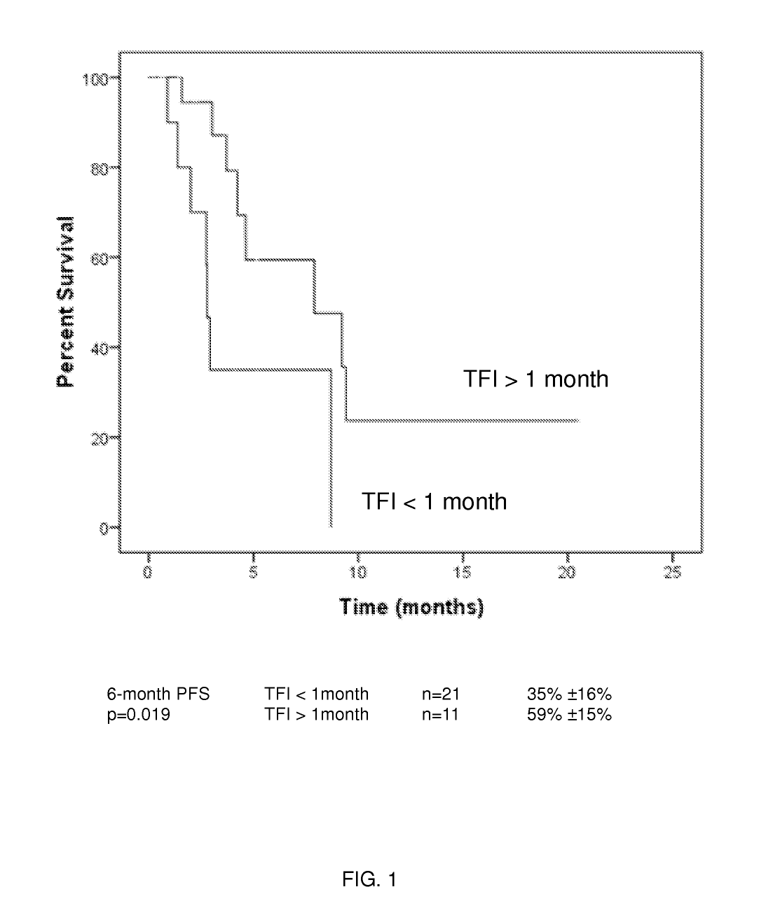

The median number of regimens prior to treatment with BC was 3. The most common drugs used in combination with bevacizumab included cyclophosphamide, doxorubicin, and platinum/taxane combination. The median time to recurrence following BC treatment was 3.3 months (range: 0.2-20.5) (Table 2). The clinical and treatment factors associated with response to BC treatment were evaluated and it was found that TFI prior to BC was associated with progression-free survival (PFS) after BC therapy. More specifically, those with TFI≧1 month prior to BC had a 6-month PFS of 59%±15% vs. 35%±16% in the group with TFI<1 month (p=0.019; see FIG. 1 and Table 3).

TABLE 2
Overall Survival based on demographic and clinical factors

| | 5-year Overall | |
|---|---|---|
| | Survival | p-value |
| Age | | 0.546 |
| <56 | 52% ± 14% | |
| ≧56 | 50% ± 14% | |
| Race | | 0.237 |
| White | 47.5% ± 10% | |
| Non-White | — | |
| Stage | | 0.157 |
| I-II | — | |
| III-IV | 46% ± 10% | |
| Grade | | 0.815 |
| 2 | 54% ± 16% | |
| 3 | 48% ± 13% | |
| Cytoreductive Surgery | | 0.777 |
| Optimal (<10 mm) | 43% 13% | |
| Suboptimal (>10 mm) | 50% ± 25% | |
| TFI after initial adjuvant therapy | | 0.001 |
| (median = 11 m) | | |
| <11 months | 21% ± 13% | |
| >11 months | 74% ± 11% | |
| Number of regimens prior to bevacizumab | | 0.665 |
| plus chemotherapy (median = 3) | | |
| ≦3 | 65% ± 15% | |
| >3 | 47% ± 12% | |
| TFI of last treatment prior to bevacizumab | 1-year overall | 0.027 |
| plus chemotherapy | survival* | |
| <1 month | 35% ± 16% | |
| >1 month | 80% ± 13% | |

*Overall survival following treatment with bevacizumab plus chemotherapy

TABLE 3
Response to bevacizumab measured by progression-
free survival post-bevacizumab

| | 6-month PFS after | |
|---|---|---|
| | bevacizumab plus | |
| | chemotherapy | p-value |
| Age | | 0.113 |
| <56 | 39% ± 15% | |
| ≧56 | 71% ± 13% | |
| Race | | 0.736 |
| White | 45% ± 12% | |
| Non-White | 50% ± 35% | |
| Stage | | 0.238 |
| I-II | 50% ± 35% | |
| III-IV | 44% ± 12% | |
| Grade | | 0.198 |
| 2 | 54% ± 16% | |
| 3 | 35% ± 17% | |
| Cytoreductive Surgery | | 0.271 |
| Optimal (<10 mm) | 40% ± 14% | |
| Suboptimal (>10 mm) | 75% ± 22% | |
| Treatment-free interval after | | 0.739 |
| initial adjuvant therapy | | |
| (median = 11 m) | | |
| <11 months | 49% ± 17% | |
| >11 months | 38% ± 16% | |
| Number of failed regimens prior | | 0.283 |
| to bevacizumab plus chemotherapy | | |
| (median = 3) | | |
| ≦3 | 57% ± 19% | |
| >3 | 38% ± 14% | |
| Treatment-free interval prior | | 0.019 |
| to bevacizumab | | |
| No | 35% ± 16% | |
| Yes | 59% ± 15% | |

The miRNA profiles associated with BC treatment response were evaluated. Using the raw data (level I) from the TCGA, 33 differentially expressed (±2.0 fold expression) miRNAs were identified (out of 800 microRNA in TGCA) including: let-7e, miR-128, miR-133a, miR-133b, miR-135b, miR-139-5p, miR-140-3p, miR-142-5p, miR-143, miR-143*, miR-144, miR-145, miR-145*, miR-15b, miR-181a, miR-183, miR-192, miR-194, miR-202, miR-21*, miR-214, miR-215, miR-222, miR-34b, miR-34b*, miR-34c-3p, miR-34c-5p, miR-375, miR-378, miR-486-5p, miR-494, miR-497, and miR-96.

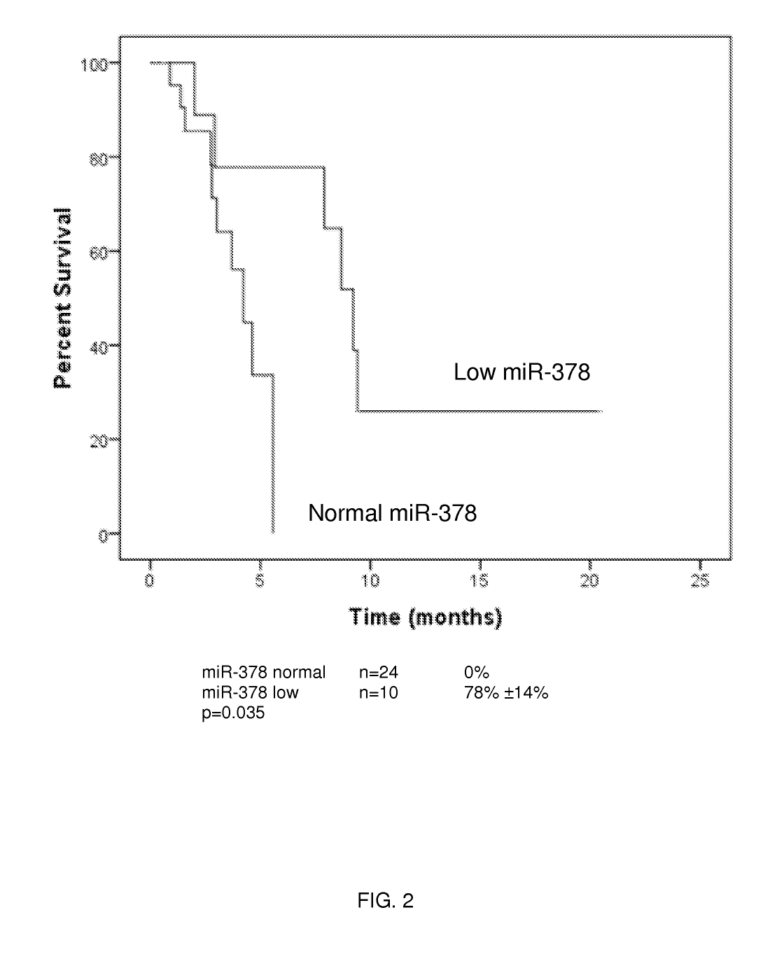

Using the interpreted data, low miR-378 (p<0.001), low miR-214 (p=0.01), high miR-128 (p=0.02), and low miR-21* (p=0.028) were found to be associated with higher proportion with 6 month PFS after BC therapy while low miR-194 (p=0.005), was correlated with lower PFS after BC. After adjusting for multiple comparisons using Bonferroni correction, miR-378 remained as a predictor for 6-month PFS after BC. More specifically, 60% of patients with low miR-378 expression had 6-month or greater PFS whereas all patients with normal miR-378 recurred or died within 6 months (p=0.005 after Bonferroni correction) (Table 4, FIG. 2).

TABLE 4
Progression-free survival after bevacizumab
and chemotherapy and MicroRNAs

| | | p-value |
|---|---|---|
| MicroRNA | % 6-month PFS after BC | (Fisher exact) |
| miR-378 | | <0.001^{#} |
| Low | 60% | |
| Normal | 0% | |
| miR-194 | | 0.005 |
| Low | 7% | |
| Normal | 67% | |
| miR-214 | | 0.01 |
| Low | 46% | |
| Normal | 5% | |
| miR-128 | | 0.02 |
| High | 50% | |
| Normal | 8% | |
| miR-21* | | 0.028 |
| Low | 36% | |
| Normal | 5% | |
| miR-375 | | 0.18 |
| High | 31% | |
| Normal | 10% | |
| miR-145* | | 0.3 |
| High | 23% | |
| Normal | 0% | |

^{#}p-value = 0.005 after Bonferroni correction

The 6-month and median PFS after BC in those with low miR-378 was 77.8% and 9.2 months compared to 0% and 4.2 months with normal miR-378 (p=0.035) (FIG. 2). On multivariate analysis, both miR-378 expression (HR=2.04, 95% CI 1.12-3.72; p=0.020) and TFI prior to BC (HR=0.75, 95% CI 0.58-0.97; p=0.030) remained as independent predictors for response to BC treatment (Tables 5 and 6).

TABLE 5
MicroRNA associated with overall survival: Kaplan-meier analysis

| | 5-year Overall | |
|---|---|---|
| MicroRNA | Survival | p-value |
| miR-145* | | 0.164 |
| High | 21% ± 19% | |
| Normal | 58% ± 11% | |
| miR-214 | | 0.173 |
| Low | 40% ± 16% | |
| Normal | 58% ± 12% | |
| miR-378 | | 0.793 |
| Low | 38% ± 16% | |
| Normal | 59% ± 12% | |
| miR-128 | | 0.468 |
| High | 83% ± 15% | |
| Normal | 48% ± 10% | |
| miR-194 | | 0.751 |
| Low | 50% ± 20% | |
| Normal | 52% ± 11% | |
| miR-21* | | 0.560 |
| Low | 56% ± 14% | |
| Normal | 44% ± 14% | |
| miR-375 | | 0.125 |
| High | 39% ± 16% | |
| Normal | 58% ± 12% | |

TABLE 6
Cox Regression model: Backward Stepwise method.

| | Hazard | 95% Confidence | |
|---|---|---|---|
| | Ratio | Interval | p-value |
| Treatment-free interval prior to | 0.754 | 0.584-0.974 | 0.030 |
| bevacizumab plus chemotherapy | | | |
| miR-378 | 2.042 | 1.116-3.974 | 0.020 |

TFI of last treatment prior to bevacizumab plus chemotherapy (months, continuous variable)
miR-378 in fold-expression, continuous variable

Further analyses were performed to explore whether miR-378 or other miRNA expression is simply a surrogate marker for overall survival. The 5-year OS of the 34 patients who underwent BC with low miR-378 vs. normal miR-378 was not different (38%±16% vs. 59%±12%; p=0.79, See Table 5). In order to demonstrate that mir-378 did not simply predict for response to chemotherapy, an additional subgroup analysis of 103 recurrent ovarian cancer patients treated with chemotherapy without bevacizumab was performed. The data showed that miR-378 did not predict for response to chemotherapy alone (2-year PFS at 31%±9% for low miR-378 vs. 7%±3% for normal miR-378; p=0.093). The corresponding 5 year OS was 17.7% vs. 17.6%; p=0.64). In addition, other miRNAs including mir-194, mir-214, and mir-128 were not associated with overall survival.

MiRNA target prediction using Targetscan 5.1 (as described in Methods) demonstrated that miR-378 targeted genes associated with tumor angiogenesis including bone morphogenetic protein 2 (BMP2), mitogen-activated protein kinase 1 (MAPK1), and Cas-Br-M ecotropic retroviral transforming sequence (CBL) amongst other genes. The context scores for BMP2, MAPK1, and CBL were -0.39, -0.34, and -0.34, respectively.

Accordingly, the results indicate that low expression levels of miR-378 confer improved survival after treatment with BC, presumably due to more effective targeting of upregulated angiogenesis in this class of patients. Low miR-214 (p=0.01), high miR-128 (p=0.02), and low miR-21* (p=0.028) were also associated with a higher 6 month PFS after BC therapy while low miR-194 (p=0.005), was correlated with lower PFS after BC.

### Example 2

### The Association of VEGF-A, -B, and -C Expression in the Survival of Serous Ovarian Cancer Patients and Response to Anti-Vascular Agents

Tumor and normal tissues from patients identified in TCGA were analyzed for VEGF expression to determine the significance of VEGF expression on the survival of serous epithelial ovarian cancer patients. VEGF expression was determined by lowess normalization and log₂ transformation to give expression values, as performed by University of North Carolina, Chapel Hill on the Agilent platform. The expression of VEGF-A, -B, and -C was determined for tumor tissue relative to normal tissue for each patient, expressed as a ratio, and the average ratio was determined for the cohort. The ratio of expression in tumor to normal tissue expression (of VEGF-A, -B, or -C) each patient was then analyzed to determine whether the expression of each VEGF was relatively greater than or relatively less than the average ratio for the cohort. Demographic, clinico-pathologic, and outcomes data were obtained from TCGA.

Specimens from all patients (483) diagnosed with serous ovarian cancer with matched genomic expression data were analyzed. The median patient age was 59 years, consisting of 92%, 5%, and 3% Whites, Black, and Asian. Stage I, II, III, and IV disease were found in 3%, 5%, 77%, and 15% of patients. The majority (86%) had grade 3 tumors, 13% had grade 2 and 1% had grade 1 disease. Of patients who underwent primary surgery, 73% were optimally debulked (<1 cm residual disease). The median follow-up time was 30 months (range: 0-180), and the five-year overall survival (OS) was 33% with median survival of 45 months. In addition, a subset of these patients (the same patient cohort who received bevacizumab, discussed in Example 1) was analyzed separately.

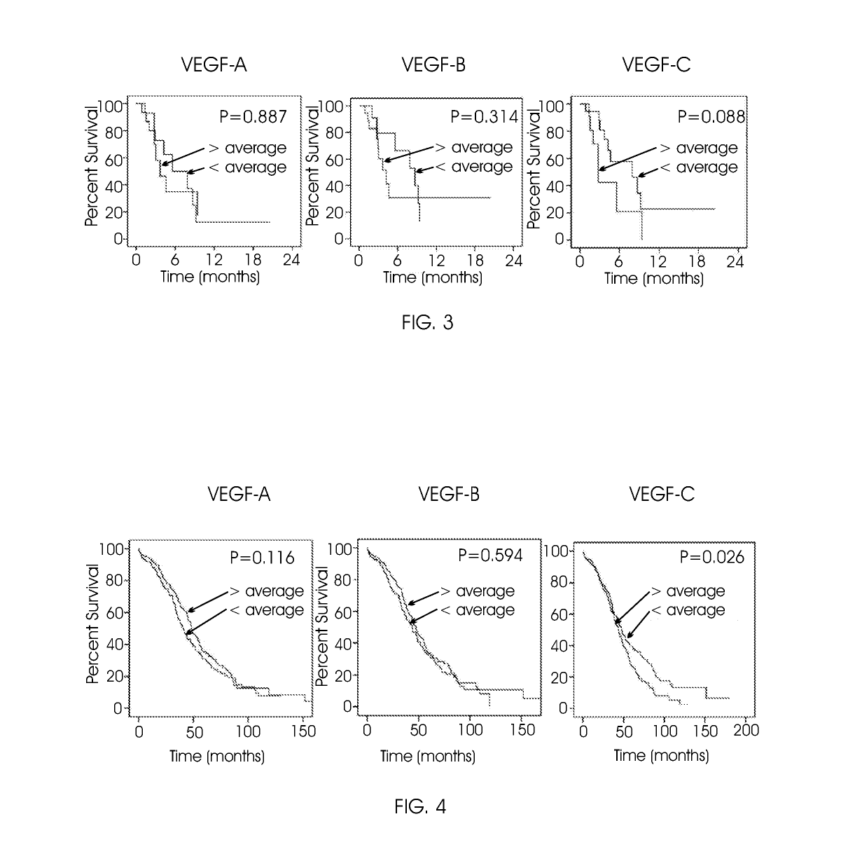

The results are shown in FIGS. 3 and 4. FIG. 3 illustrates the progression-free survival of the patient cohort treated with bevacizumab according to whether the tumor expression of VEGF-A, -B, or -C (relative to normal tissue expression) was relatively greater than or relatively less than the average ratio for the cohort. FIG. 4 illustrates the progression-free survival of patients in the entire cohort having relatively greater than or relatively less than average expression of VEGF-A, -B, or -C (relative to normal tissue expression), respectively.

As shown in FIG. 4, patients with high expression of VEGF-C (tumor tissue relative to normal tissue expression) had worse OS than those with low VEGF-C (median 48 months vs. 43 months, 5-year OS 40% vs. 27%, respectively; p=0.026). VEGF-A and VEGF-B were not significantly associated with prognosis. Earlier stage (p=0.003) and white race (p=0.028) were associated with better survival. On multivariate analyses, race (HR=1.23; 95% CI: 1.05-1.44; p=0.009) and stage (HR=1.46; 95% CI: 1.13-1.89; p=0.003) remained as important prognosticators.

In a subset analysis of those who underwent treatment with bevacizumab, there was a suggestion that higher expression of VEGF-C was associated with poorer response, suggesting that this subset of patients exhibits a poorer response to anti-vascular agents. Due to the small sample size (34 patients), the bevacizumab treated patient population did not reach statistical significance.

It was concluded that the expression of VEGF-C could be used as a prognostic biomarker for ovarian cancer survival and response to anti-vascular agents.

### Example 3

### Immunoregulation Gene Expression and Response to Bevacizumab in Ovarian Cancer

To determine the association of immune marker expression and response to bevacizumab in recurrent serous ovarian cancer, clinical and genomic data were obtained from TCGA data portal and analyzed. Kaplan-Meier survival estimates and Cox proportional-hazards model were employed for statistical analyses.

All patients had recurrent serous ovarian cancer. The median time to primary recurrence of 10.9 months (range: 0.2-101.5 months) and 5-year overall survival (OS) was 47.4% with a median survival of 57.4 months. A total of 32 patients matched with gene expression were treated with bevacizumab combined with chemotherapy (BC). The most common drugs used in combination with bevacizumab included cyclophosphamide, doxorubicin, and platinum/taxane combination. The median time to recurrence following BC treatment was 4.6 months (range: 0.2-20.5). Longer treatment-free interval prior to BC treatment was associated with better response; patients with greater than 1 month TFI had 7.9 months of median PFS compared to 2.8 months in the group with shorter TFI.

A list of candidate genes that regulate immune function was evaluated to identify associations with BC response. A low expression of CCL2 and high expression of CD55 were associated with longer PFS after BC. Kaplan-Meier estimates showed patients with low CCL2 had a 6-month and median PFS of 68% and 8.7 months compared to 20% and 2.9 months (p=0.009). Those with high CD55 showed an improved response to BC (6-month PFS 59% and 8.7 months median PFS vs. 25% and 3.0 months; p=0.03).

On multivariate analysis, TFI (HR 0.23, 95% CI 0.06-0.84; p=0.025) was an independent predictor for BC response. Tumors expressing CD55 had a decreased risk of progression (HR=0.39, 95% CI 0.15-1.01; p=0.052).

These data suggest that immune-modulation may be associated with treatment response after bevacizumab with chemotherapy in recurrent ovarian cancer.

### REFERENCES

1. Folkman J. Role of angiogenesis in tumor growth and metastasis. Seminars in oncology 2002; 29:15-8.
2. Ranpura V, Hapani S, Wu S. Treatment-related mortality with bevacizumab in cancer patients: a meta-analysis. JAMA 2011; 305:487-94.
3. Verheul H M, Lolkema M P, Qian D Z, et al. Platelets take up the monoclonal antibody bevacizumab. Clin Cancer Res 2007; 13:5341-7.
4. Grothey A, Galanis E. Targeting angiogenesis: progress with anti-VEGF treatment with large molecules. Nat Rev Clin Oncol 2009; 6:507-18.
5. Ivy S P, Wick J Y, Kaufman B M. An overview of small-molecule inhibitors of VEGFR signaling. Nat Rev Clin Oncol 2009; 6:569-79.
6. Heath V L, Bicknell R. Anticancer strategies involving the vasculature. Nat Rev Clin Oncol 2009; 6:395-404.
7. Hsu J Y, Wakelee H A. Monoclonal antibodies targeting vascular endothelial growth factor: current status and future challenges in cancer therapy. BioDrugs 2009; 23:289-304.
8. Gille J. Antiangiogenic cancer therapies get their act together: current developments and future prospects of growth factor- and growth factor receptor-targeted approaches. Exp Dermatol 2006; 15:175-86.
9. Jemal A, Siegel R, Ward E, Hao Y, Xu J, Thun M J. Cancer statistics, 2009. CA: a cancer journal for clinicians 2009; 59:225-49.
10. Zhao Y, Samal E, Srivastava D. Serum response factor regulates a muscle-specific microRNA that targets Hand2 during cardiogenesis. Nature 2005; 436:214-20.
11. Chan J A, Krichevsky A M, Kosik Kans. MicroRNA-21 is an antiapoptotic factor in human glioblastoma cells. Cancer research 2005; 65:6029-33.
12. Cimmino A, Calin G A, Fabbri M, et al. miR-15 and miR-16 induce apoptosis by targeting BCL2. Proc Natl Acad Sci USA 2005; 102:13944-9.
13. Costinean S, Zanesi N, Pekarsky Y, et al. Pre-B cell proliferation and lymphoblastic leukemia/high-grade lymphoma in E(mu)-miR155 transgenic mice. Proc Natl Acad Sci USA 2006; 103:7024-9.
14. Croce C M, Calin G A. miRNAs, cancer, and stem cell division. Cell 2005; 122:6-7.
15. Mello C C, Czech M P. Micromanaging insulin secretion. Nat Med 2004; 10:1297-8.
16. Triboulet R, Mari B, Lin Y L, et al. Suppression of microRNA-silencing pathway by HIV-1 during virus replication. Science 2007; 315:1579-82.
17. Chellappan P, Vanitharani R, Fauquet C M. MicroRNA-binding viral protein interferes with Arabidopsis development. Proc Natl Acad Sci USA 2005; 102:10381-6.
18. Merritt W M, Lin Y G, Han L Y, et al. Dicer, Drosha, and outcomes in patients with ovarian cancer. N Engl J Med 2008; 359:2641-50.
19. Lee D Y, Deng Z, Wang C H, Yang B B. MicroRNA-378 promotes cell survival, tumor growth, and angiogenesis by targeting SuFu and Fus-1 expression. Proc Natl Acad Sci USA 2007; 104:20350-5.
20. Hwang J H, Voortman J, Giovannetti E, et al. Identification of microRNA-21 as a biomarker for chemoresistance and clinical outcome following adjuvant therapy in resectable pancreatic cancer. PLoS One; 5:e10630.
21. Ranade A R, Cherba D, Sridhar S, et al. MicroRNA 92a-2*: a biomarker predictive for chemoresistance and prognostic for survival in patients with small cell lung cancer. J Thorac Oncol; 5:1273-8.
22. Hummel R, Hussey D J, Haier J. MicroRNAs: predictors and modifiers of chemo- and radiotherapy in different tumour types. Eur J Cancer; 46:298-311.
23. Sherman A E H L, Melton C, Lu T, Blelloch R, Chan J K. The association of microRNA mir-21 in ovarian cancer drug resistance. Gynecol Oncol 2009; In Press.
24. Slack F J, Weidhaas J B. MicroRNA in cancer prognosis. N Engl J Med 2008; 359:2720-2.
25. Eitan R, Kushnir M, Lithwick-Yanai G, et al. Tumor microRNA expression patterns associated with resistance to platinum based chemotherapy and survival in ovarian cancer patients. Gynecol Oncol 2009; 114:253-9.
26. Nam E J, Yoon H, Kim S W, et al. MicroRNA expression profiles in serous ovarian carcinoma. Clin Cancer Res 2008; 14:2690-5.
27. Heusschen R, van Gink M, Griffioen A W, Thijssen V L. MicroRNAs in the tumor endothelium: novel controls on the angioregulatory switchboard. Biochim Biophys Acta 2010; 1805:87-96.
28. Fish J E, Srivastava D. MicroRNAs: opening a new vein in angiogenesis research. Sci Signal 2009; 2:pe1.
29. Qiu H, Yang B, Pei Z C, Zhang Z, Ding K. WSS25 inhibits growth of xenografted hepatocellular cancer cells in nude mice by disrupting angiogenesis via blocking bone morphogenetic protein (BMP)/Smad/Id1 signaling. J Biol Chem; 285:32638-46.
30. Bieniasz M, Oszajca K, Eusebio M, Kordiak J, Bartkowiak J, Szemraj J. The positive correlation between gene expression of the two angiogenic factors: VEGF and BMP-2 in lung cancer patients. Lung Cancer 2009; 66:319-26.
31. Brunckhorst M K, Wang H, Lu R, Yu Q. Angiopoietin-4 promotes glioblastoma progression by enhancing tumor cell viability and angiogenesis. Cancer Res; 70:7283-93.
32. LaRusch G A, Mandi F, Shariat-Madar Z, et al. Factor XII stimulates ERK1/2 and Akt through uPAR, integrins, and the EGFR to initiate angiogenesis. Blood; 115:5111-20.
33. Deregibus M C, Buttiglieri S, Russo S, Bussolati B, Camussi G. CD40-dependent activation of phosphatidylinositol 3-kinase/Akt pathway mediates endothelial cell survival and in vitro angiogenesis. J Biol Chem 2003; 278:18008-14.
34. Holmqvist K, Welsh M, Lu L. A role of the protein Cbl in FGF-2-induced angiogenesis in murine brain endothelial cells. Cell Signal 2005; 17:1433-8.
35. Wang S, Olson E N. AngiomiRs—key regulators of angiogenesis. Curr Opin Genet Dev 2009; 19:205-11.
36. Meng Z, Fu X, Chen X, et al. miR-194 is a marker of hepatic epithelial cells and suppresses metastasis of liver cancer cells in mice. Hepatology 2010; 52:2148-57.
37. Sen C K, Gordillo G M, Khanna S, Roy S. Micromanaging vascular biology: tiny microRNAs play big band. J Vasc Res 2009; 46:527-40.
38. Chan L S, Yue P Y, Mak N K, Wong R N. Role of microRNA-214 in ginsenoside-Rg1-induced angiogenesis. Eur J Pharm Sci 2009; 38:370-7.
39. Kaplan E L, Meier P. Nonparametric estimation from incomplete observations. J Am Stat Assoc 1958; 53:457-81.
40. Cox D R. Regression models and life tables. Journal of the Royal Statistical Society: Series B 1972; 34:187-220.
41. Grimson A, Farh K K, Johnston W K, Garrett-Engele P, Lim L P, Bartel D P. MicroRNA targeting specificity in mammals: determinants beyond seed pairing. Mol Cell 2007; 27:91-105.
42. Chambers, S. K., et al., Overexpression of tumor vascular endothelial growth factor A may portend an increased likelihood of progression in a phase II trial of bevacizumab and erlotinib in resistant ovarian cancer. Clin. Cancer Res. 2010; 16:5320
43. White, N. M. A., et al. Metastamirs: a stepping stone towards improved cancer management Nat. Rev. Clin. Onc. 2011; 8:75
44. Reichert, J. M. Antibody-based therapeutics to watch in 2011 MAbs 2011; 3:76.
45. Karlan, B. Y., et al. Randomized, double-blind, placebo-controlled phase II study of AMG 386 combined with weekly paclitaxel in patient with recurrent ovarian cancer J. Clin Oncol. 2012; 30:362.
46. Perren, T. J., et al. A phase 3 trial of bevacizumab in ovarian cancer NEJM 2011; 365: 2482.
47. Burger, R. A. et al., Incorporation of bevacizumab in the primary treatment of ovarian cancer NEJM 2011; 365:2473.

What is claimed is:

1. A method for selecting a patient for treatment with an anti-angiogenic agent comprising the steps:
obtaining samples of tumor and normal tissues from a patient;
determining the miRNA or protein expression in said samples;
comparing the miRNA or protein expression for each miRNA or protein in said sample of tumor tissue to the miRNA or protein expression in said sample of normal tissue;
wherein if the miRNA or protein expression indicates that the tumor tissue exhibits upregulated angiogenesis, the patient is scheduled for treatment with said anti-angiogenic agent.

2. The method of claim 1, wherein the expression of miR-378, miR-214 or miR-21 is low in said sample of tumor tissue relative to normal tissue.

3. The method of claim 1, wherein the expression of miR-128 or miR-194 is high in said sample of tumor tissue relative to normal tissue.

4. The method of claim 1, wherein the expression of VEGF-C is high in said sample of tumor tissue relative to normal tissue.

5. The method of claim 1, further comprising treating the patient with an anti-angiogenic agent.

6. The method of claim 1, wherein said patient is suffering from cancer.

7. The method of claim 2, wherein said cancer is selected from brain, breast, kidney, pancreatic, lung, prostate, colorectal, and ovarian cancers.

8. The method of claim 1, wherein said anti-angiogenic agent is selected from a VEGF-targeting agent or a tyrosine kinase inhibitor.

9. The method of claim 1, wherein said VEGF-targeting agent is bevacizumab.

10. A method for treating a patient suffering from cancer with an anti-angiogenic agent, comprising
obtaining samples of tumor and normal tissues from a patient;
determining the miRNA or protein expression for angiogenic genes in said samples;
comparing the miRNA or protein expression for angiogenic genes for each miRNA or protein in said sample of tumor tissue to the miRNA or protein expression in said sample of normal tissue;
wherein if said miRNA expression indicates that the expression of at least one angiogenic gene is upregulated in tumor tissue, the patient is treated with an anti-angiogenic agent.

11. The method of claim 10, wherein said anti-angiogenic agent is selected from a VEGF-targeting agent, or a tyrosine kinase inhibitor.

12. The method of claim 10, wherein the expression of miR-378, miR-214 or miR-21 is low in said sample of tumor tissue relative to normal tissue.

13. The method of claim 10, wherein the expression of miR-128 or miR-194 is high in said sample of tumor tissue relative to normal tissue.

14. The method of claim 10, wherein the expression of VEGF-C is high in said sample of tumor tissue relative to normal tissue.

15. The method of claim 10, wherein said angiogenic gene is selected from bone morphogenetic protein 2 (BMP2), mitogen-activated protein kinase 1 (MAPK1), or Cas-Br-M ecotropic retroviral transforming sequence (CBL).

16. A method for reducing the risk of adverse events from treatment with an anti-angiogenic agent in a population of patients suffering from cancer comprising
obtaining a sample of normal tissue and a sample of tumor tissue from a patient;
determining the expression of miRNA or protein in said samples;
comparing the expression of each miRNA or protein in the tumor sample to levels of expression of each miRNA or protein in normal tissue;
wherein if one of the following expression of miRNA or protein is found, the patient is scheduled for treatment with an anti-angiogenic agent:
miR-378, miR-214 or miR-21 is low in said tumor sample relative to said normal sample;
miR-128 or miR-194 is high in said tumor sample relative to said normal sample;
VEGF-C expression is high in said tumor sample relative to said normal sample;
complement inhibitor expression is high in said tumor sample relative to said normal sample; or
inflammatory chemokine expression is low in said tumor sample relative to said normal sample.

17. The method of claim 16, wherein the inflammatory chemokine is CCL2.

18. The method of claim 16, wherein the complement inhibitor is CD55.

19. The method of claim 16, wherein said anti-angiogenic agent is selected from a VEGF-targeting agent or a tyrosine kinase inhibitor.

20. The method of claim 19, wherein said VEGF-targeting agent is bevacizumab.
